(19) United States
(12) Patent Application Publication
(10) Pub. No.: US 2024/0182664 A1
(43) Pub. Date: Jun. 6, 2024

(54) Method For The Depolymerization Of Polycaprolactam Processing Waste To Form Caprolactam

(71) Applicant: IONIQA TECHNOLOGIES B.V., Eindhoven (NL)

(72) Inventors: Luuk Lambertus Christina Olijve, Eindhoven (NL); Michael Josef DE GROOT, Eindhoven (NL); Andre Banier DE HAAN, Eindhoven (NL)

(73) Assignee: Ioniqa Technologies B.V., Eindhoven (NL)

(21) Appl. No.: 18/284,366

(22) PCT Filed: Mar. 31, 2022
(86) PCT No.: PCT/NL2022/050180

§ 371 Date: Sep. 27, 2023

(30) Foreign Application Priority Data
Apr. 1, 2021 (NL) 2027900

(51) Int. Cl. C08J 11/20; C07D 225/02

(52) U.S. Cl. CPC C08J 11/20; C07D 225/02; C08J 2377/02

(57) ABSTRACT

A continuous method for depolymerizing polycaprolactam waste into caprolactam is described. In the method, a melt of the polycaprolactam waste and an inert gas are fed to a reactor in a continuous manner. Water and an aromatic hydrocarbon are also fed to the reactor and the polycaprolactam waste is contacted with superheated steam of the water/hydrocarbon mixture at a temperature of between 260° C. and 300° C. and at a gauge pressure from 1 barg to 70 barg. Turbulent mixing conditions are created in the reactor, and a caprolactam-containing vapor stream is created in the reactor which exits the reactor at an outlet. The caprolactam is separated from the exited caprolactam-containing vapor stream by partial condensation, and collected. A reactor system for carrying out the method is also described.

### FIELD OF THE INVENTION

The present invention relates to a method for the depolymerization of polycaprolactam processing waste to form caprolactam. Polycaprolactam is also referred to in this application as nylon 6 or polyamide 6 (PA6).

### BACKGROUND OF THE INVENTION

There is an increasing awareness that the large amount of polymers used nowadays for a variety of purposes should be recycled in order to prevent an increasing amount of polymer waste. Incineration is one possibility, however undesirable for obvious reasons. Mechanical shredding and milling of used polymers may be another solution to the problem of accumulating polymer waste. However, the recycled polymer properties are then degraded and they frequently end up as filler for other materials. Circular recycling (chemical recycling) is clearly the solution of choice. In circular recycling, the polymer to be recycled is depolymerized into its building blocks, such as the repeating units from which the polymer is made. The repeating units resulting from the degradation reaction may be used again in making a new polymer.

Degradation of used polymers may be hindered by the fact that such polymers are typically present in a product that may contain a plurality of materials, and suitable separation methods should be provided for separating the polymer to be recycled from the product. As a consequence a significant amount of used polymers is still used as a fuel, which is burned. Polymers may comprise fillers and other additives to enhance their properties. They may be natural or man-made. They may also be combined with another polymer in a polymer blend, or form a copolymer. Combining polymers with other materials such as metals is also possible, such as in fiber-metal laminates. All this makes recycling more difficult.

A further difficulty seems to be assuring a consistent and continuous waste sourcing, such as fibers, films, bottles and textiles for instance, in a required extensive amount at one single site. Yet a further issue may be that, even when the problem of separation of the desired polymer to be recycled from a product has been solved satisfactorily, the process of degradation of the polymer into smaller repeating units, such as its repeating units, has proven to be difficult. Many known methods are not selective enough, or are deficient in terms of a too low conversion. An efficient conversion of the polymers to the desired products (repeating units and/or oligomers) is desirable, at the same time minimizing production of waste in terms of side products. In other words, a relatively high yield (selectivity times conversion) is a desirable goal in depolymerization methods.

Nylon 6 is one of the most prominent polymers used nowadays. It is typically processed into a variety of intermediate articles such as fibers, textiles, carpets, chips, films, an/or end products such as fabrics, carpets, packaging and molded articles. The processing of polycaprolactam into intermediate articles and end products results in a huge amount of nylon 6 processing waste, i.e. in the form of scrap nylon 6 polymer and/or oligomers.

For nylon 6 also, circular recycling methods have been developed. Such methods aim at depolymerizing scrap nylon 6 materials back to the caprolactam monomer, which caprolactam monomer may then be reused in forming nylon 6 again by polymerization.

In general, nylon 6 is depolymerized by heating at elevated temperatures, usually in the presence of a catalyst and/or steam. U.S. Pat. No. 5,869,654 for instance discloses a method wherein superheated steam and melted scrap nylon 6 are combined in a tubular elongated reactor. The method comprises feeding a melt of the polycaprolactam waste to a reactor in a continuous manner; in the absence of added catalyst, contacting said polycaprolactam waste with superheated steam at a temperature of 250° C. to 400° C. and at a pressure within the range of 1.5 atm to 100 atm and less than the saturated vapor pressure of water at said temperature; wherein said contacting occurs counter-currently or cross-currently with superheated steam, and wherein a caprolactam-containing vapor stream is formed, from which stream the caprolactam is recovered by condensation. U.S. Pat. No. 5,869,654 further teaches that temperatures of at least 250° C. are preferred because below 250° C., caprolactam formation may be too slow, whereas temperatures no greater than 400° C. are preferred, as above 400° C. side reactions of nylon 6 may become prohibitively fast. A most preferred temperature range is from 300° C. to 340° C. Only at these relatively high temperatures, acceptable caprolactam yields are obtained. Example 1 of U.S. Pat. No. 5,869,654 indeed shows that when maintaining a temperature and pressure in the reactor at about 312° C. and 9.2 atm respectively, it is possible to achieve a normalized rate of caprolactam production of about 0.7 to 0.8 g caprolactam per gram of nylon 6 melt held in the reactor per hour.

EP 1975156A1 discloses a static process for depolymerizing nylon 6 using a mixture of water and a hydrocarbon. The process has to be carried out at a temperature above 300° C. to obtain reasonable caprolactam yields.

Although the caprolactam yield of the known process is acceptable, there is a need in the industry for an improved method for the depolymerization of polycaprolactam processing waste.

### SUMMARY OF THE INVENTION

The invention thereto provides a continuous method for depolymerizing polycaprolactam waste in accordance with claim 1. The method in particular comprises the steps of:
a) feeding a melt of the polycaprolactam waste and an inert gas to a reactor in a continuous manner;
b) feeding water and a hydrocarbon having a boiling point at atmospheric pressure of between 80° C. and 270° C. to an inlet of the reactor and contacting the polycaprolactam waste with superheated vapor steam of the water/hydrocarbon mixture at a temperature of between 260° C. and 300° C. and at a gauge pressure from 1 barg to 70 barg;
c) creating turbulent mixing conditions in the reactor;
d) forming a caprolactam-containing vapor stream in the reactor which exits the reactor at an outlet;
e) separating caprolactam from the exited caprolactam-containing vapor stream by partial condensation; and
f) collecting the caprolactam from said condensed caprolactam-containing vapor stream.

According to the invention, said water/hydrocarbon mixture comprises at least 1 wt. % and at most 40 wt. % water, relative to the total weight of the water/hydrocarbon mixture, and the hydrocarbon is aromatic in that it comprises benzene and/or benzene derivatives having from one to six substituents attached to the central benzene core.

The turbulent mixing conditions are preferably created in the reactor by feeding the water and a hydrocarbon into the reactor.

The prior art method uses water to create the vapor stream in the reactor. It has turned out that by replacing a substantial part of the water with a hydrocarbon, the reaction balance is more favorable to the caprolactam monomer and a lower reaction temperature of between 260° C. and 300° C. becomes possible for stripping caprolactam from nylon 6 waste at a high yield, such as above 85%.

Apart from saving on capital expenditure, the possibility of using lower temperatures than known in the art also avoids or hinders side reactions and decomposition of the formed 6-amino-caproic acid (ACA) intermediate as well as undesired formation of ammonia.

In the method, the superheated vapor stream effectively hydrolyses the supplied molten nylon 6. The overheated steam is typically supplied to an underside of the reactor, and caprolactam is stripped and condensed through an overhead of the same reactor. According to the known method, a higher temperature and a higher amount of water cause a higher degree of caprolactam recovery. The known method requires a relatively high reactor pressure to ensure a longer residence time of water in the reactor.

The presently claimed method avoids such disadvantages of the known method, and still yields reduced ammonia and dimer formation. Too high temperatures exceeding 300° C. may cause decomposition and ammonia formation of probably the 6-amino caproic acid (ACA) intermediate. Secondly, too much water ensures that the reaction balance lies with the ACA instead of with caprolactam. This increases the likelihood of decomposition and side reactions. In the lactam form, the amine group is better protected from decomposition. Thirdly, a too low vapor steam to PA6 ratio and a too low vapor steam mass flow (and thus a longer residence time) probably cause a slow caprolactam recovery kinetics), which also leads to a greater chance of decomposition of the monomer. Indeed, the caprolactam slowly disappears from the reactor because the vapor steam acts as carrier for the caprolactam formed. It is surprising that a caprolactam yield exceeding 85% may be obtained at temperatures below 300° C., and/or gauge pressures within the range of 10-70 barg. It is a general believe that a temperature of at least 300° C. is needed for a caprolactam yield of more than 90%. At lower temperatures of 280° C. for instance, the yield is expected to be much lower, for instance in the order of 50-60% after several hrs. The presently claimed invention is able to achieve a yield of 87% and more after 1 hr only. Typical reaction times are from 30 min to 2 hrs, more preferably from 45 min to 1.5 hrs. Preferably, the reaction temperature is selected to a temperature that is just above the boiling point of caprolactam (b.p.=270° C.), i.e. within the temperature range from the boiling point of caprolactam to 30° C. above said boiling point, more preferably to 20° C. above said boiling point, even more preferably to 10° C. above said boiling point.

An essential feature of the invention is that turbulent mixing conditions should be created in the reactor. This is achieved by adding the hydrocarbon apart from water. Providing mixing means in the reactor promotes transport and distribution of the PA6 polymer in the reactor. Rotation speeds may vary according to the circumstances but a preferred range of rotation speeds is from 10-1000 RPM, more preferably from 200-800 RPM, and most preferably from 300-600 RPM for one mixing means, such as a rotating assembly of a plurality of blades, preferably provided in a lower section of one reactor, more preferably within a lower half height of the reactor, and even more preferably within a lower quarter height of the reactor. The turbulent mixing caused by adding the hydrocarbon ensures a required level of mixing of the water/hydrocarbon/PA 6 melt. If a good mixing is not obtained, the PA6 polymer will melt at the reaction temperature but the water/hydrocarbon mixture will have the tendency to remain at a relatively low position close to the bottom of the reactor as a separate phase. The mixing means helps in distributing the PA6 polymer melt.

According to the invention, the superheated vapor steam comprises a hydrocarbon, apart from water. The hydrocarbon added to the water acts as an inert dilutant of the reaction mixture. It ensures that the PA6 melt is diluted to a lower viscosity, and that the water concentration is lower, which, as said, promotes lactam formation because the reaction balance has been shifted more favorably to caprolactam. Due to the lower viscosity of the reaction mixture, turbulent stirring conditions are more easily achieved, which enhances yield. The hydrocarbon also functions as an inert carrier, which causes the caprolactam to be stripped out of the reactor faster. This promotes caprolactam recovery kinetics and reduces the chance of decomposition because caprolactam is removed from the reactor faster. The added hydrocarbon also functions as a heat-transfer medium ensuring a better heat distribution and faster melting of the PA6 waste. The melting point of PA6 is around 210° C., but dissolved in a hydrocarbon according to the invention, it solvolyses around 150° C. already. The turbulent mixing conditions promote an improved mixing of the PA6 melt with the hydrocarbon and the water. This better mixing causes the water to be better distributed in the PA6 melt which enhances the hydrocatalytic action of the water. The hydrocarbon aids in achieving a lower viscous polymer solution instead of a high viscous polymer melt.

Suitable hydrocarbons have a boiling point at atmospheric pressure of between 100° C. and 270° C. in order to enhance efficient condensation of caprolactam. In preferred embodiments of the method, the hydrocarbon has a boiling point of less than 200° C., more preferably of less than 160° C., even more preferably of less than 140° C., and, most preferably around the boiling point of water at atmospheric pressure.

Other preferred embodiments relate to methods wherein the hydrocarbon has an auto-ignition temperature of more than 200° C., more preferably of more than 300° C., even more preferably of more than 400° C.

The hydrocarbon according to the invention is aromatic and comprises benzene and/or benzene derivatives having from one to six substituents attached to the central benzene core. Polycyclic aromatic hydrocarbons are excluded. Preferred methods use hydrocarbons that are bio-based.

According to an embodiment of the invention, a method is provided wherein the hydrocarbon comprises an alkylaromatic hydrocarbon. Preferred examples are those wherein the alkyl of the alkylaromatic hydrocarbon comprises methyl, ethyl, propyl, butyl or pentyl. The best results have been obtained with an embodiment of the method wherein the alkylaromatic hydrocarbon has one substituent only, such as in toluene and ethylbenzene. Other preferred embodiments comprise alkylaromatic hydrocarbons having two substituents, such as in xylene and its isomers (o-xylene, m-xylene and p-xylene or mixtures thereof). Most preferred is toluene.

Methods according to preferred embodiments carry out the hydrolysis of the polycaprolactam at a temperature of between 260° C. and 300° C., more preferably of between 270° C. and 290° C., and most preferably of between 275° C. and 285° C.

In other preferred embodiments of the method, said gauge pressure in the reactor is from 3 to 60 barg, more preferably from 5 to 40 barg, and most preferably from 10 to 30 barg.

The water/hydrocarbon mixture may contain more water than hydrocarbon. In preferred embodiments however, a method is provided wherein said water/hydrocarbon mixture comprises at most 30 wt. % water, even more preferably at most 20 wt. % water, and most preferably at most 15 wt. % water, relative to the total weight of the water/hydrocarbon mixture.

A minimum amount of water in the water/hydrocarbon mixture may be required. In preferred embodiments, methods are provides wherein said water/hydrocarbon mixture comprises at least 2 wt. % water, even more preferably at least 4 wt. % water, and most preferably at least 5 wt. % water, relative to the total weight of the water/hydrocarbon mixture.

The lower operating temperature of between 250° C. and 300° C. (end point not included) instead of an operating temperature in excess of 300° C., for instance between 300-340° C., significantly reduces the likelihood of decomposition and also allows cheaper reactor parts to be used. Further, due to the lower specific heat capacity of the claimed hydrocarbon and in particular toluene compared to water, a higher overall mass flow can be applied for hydrocarbon/water mixtures at equal boiler capacity. The higher overall mass flow through the reactor may promote the kinetics of caprolactam recovery. In other words, the overall energy consumption of the system may be reduced with equal reactor input mass flow. Still further, it has turned out that the condenser efficiency for caprolactam recovery is higher when using a hydrocarbon/water mixture instead of water, in particular when using a toluene/water mixture.

In another aspect of the invention, a continuous reactor system is provided for depolymerizing polycaprolactam waste into caprolactam. The reactor system comprises
first feeding means for feeding a melt of the polycaprolactam waste to a reactor in a continuous manner;
second feeding means for feeding an inert gas to the reactor in a continuous manner;
third feeding means for feeding water and a hydrocarbon having a boiling point at atmospheric pressure of between 100° C. and 270° C. to an inlet of the reactor and creating turbulent mixing conditions in the reactor;
heating means for heating the reactor to a temperature of between 250° C. and 300° C.; and pressure means for pressurizing the reactor to a gauge pressure from 1 barg to 70 barg, such that the polycaprolactam waste is contacted with superheated steam of the water/hydrocarbon mixture in the reactor;
mixing means provided in the reactor;
a reactor outlet for exiting a caprolactam-containing vapor stream formed in the reactor;
separating means for separating caprolactam from the exited caprolactam-containing vapor stream by partial condensation; and
collecting means for collecting the caprolactam from said condensed caprolactam-containing vapor stream.

For the manufacturing of PA6 from caprolactam, a hydrocatalytic polymerization process is generally used by means of a VK tube. A VK tube is a long reactor with different stages of temperature, pressure and water concentration. In general, the reaction temperature is between 260-290° C. At the reaction temperature, water acts as a catalyst for opening the lactam ring, and PA6 is produced via an amino acid intermediate product, as shown below:

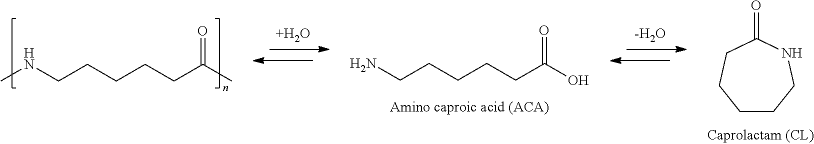

When depolymerizing PA6 into caprolactam, it is important to shift the reaction balance the other way, by adding more water and by continuously removing caprolactam from the reaction mixture. Preferably therefore, an open head reactor is used wherein the superheated vapor stream is passed through the PA6 melt (contacts the PA6) at the claimed temperature range, and caprolactam is continuously condensed via the overhead steam. A preferred reactor system allows performing a method wherein said contacting occurs in counter current flow with the superheated vapor stream in a vertical tubular reactor, preferably with an open head.

By replacing part of the water with an inert hydrocarbon such as toluene, the caprolactam yield is surprisingly increased with respect to using water, at least partly because the reaction balance is shifted favorably, and turbulent mixing conditions are favored by the addition of the hydrocarbon. A stirred continuous reactor may be used for transporting and/or distributing the PA6 polymer melt in the reactor.

### BRIEF DESCRIPTION OF THE DRAWINGS

The invention will now be described in more detail with reference to the figures, in which:

FIG. 1 schematically illustrates a continuous reactor system used in the method according to an embodiment of the invention;

FIG. 2 schematically illustrates a reactor system according to yet another embodiment of the invention;

FIG. 3 schematically illustrates a reactor system according to yet another embodiment of the invention; and

FIG. 4 schematically illustrates a reactor system according to yet another embodiment of the invention.

It should be noted that the same reference numbers in the figures are used for the same reactor system components, unless noted otherwise.

### DETAILED DESCRIPTION OF PREFERRED EMBODIMENTS

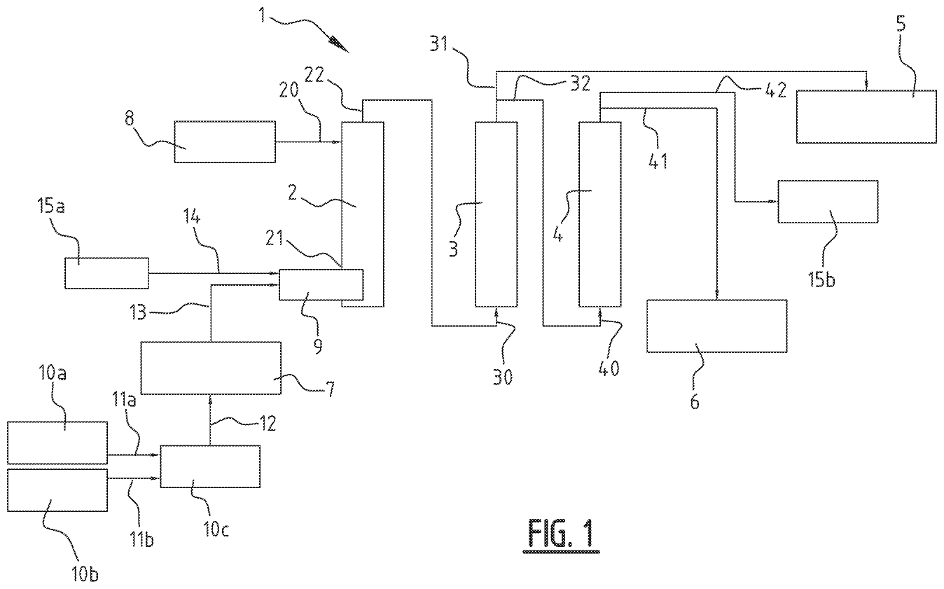

With reference to FIG. 1, a schematic diagram of a representative embodiment of a reactor system 1 and method for depolymerizing PA6 is shown. The reactor system 1 comprises a vertical stirred reactor 2, two condensers (3, 4) a caprolactam product tank 5, a solvent tank 6 and a boiler 7. A melt of PA6 is transported by first feeding means in the form of a gear pump or extruder 8 and enters the stirred reactor 2 through an inlet 20, provided at a top portion of the reactor 2. A bottom inlet 21 is provided to the stirred reactor 2 through which an inert gas such as nitrogen gas (N2) and superheated vapor are introduced in the stirred reactor 2. This is preferably done through a sparger 9, provided at a bottom portion of the reactor 2. The caprolactam contained in a vapor stream exits the stirred reactor 2 at a top portion thereof through exit 22. It then enters a first condenser 3 through inlet 30 and that connects to the caprolactam product tank 5 through an outlet 31, and to the second condenser 4 through a top or side outlet 32. Solvent, inert gas and other impurities enter the second condenser 4 at an inlet 40 thereof. Condensed solvent exits the second condenser 4 through an outlet 41 and is collected in the solvent tank 6. Inert gas exits the second condenser 4 through an outlet 42 and is collected in an inert gas tank 15b.

In the embodiment shown in FIG. 1, water and toluene are lead from their respective water and toluene tanks (10a, 10b) to a mixing unit 10c through separate conduits (11a, 11b). The water and toluene are thoroughly mixed in the mixing unit 10c and led to the boiler 7 through conduit 12. In the boiler 7, the water/toluene mixture is brought to a temperature above the highest boiling point of the two constituents and pressure to produce superheated vapor and the ensuing water/toluene vapor is led through conduit 13 to the sparger 9. An inert gas such as N₂ that originates from inert gas tank 15a is also fed to the sparger 9 through conduit 14. The water/toluene vapor mixture and the inert gas combine in the sparger 9 and are introduced in the reactor 2 under turbulent conditions.

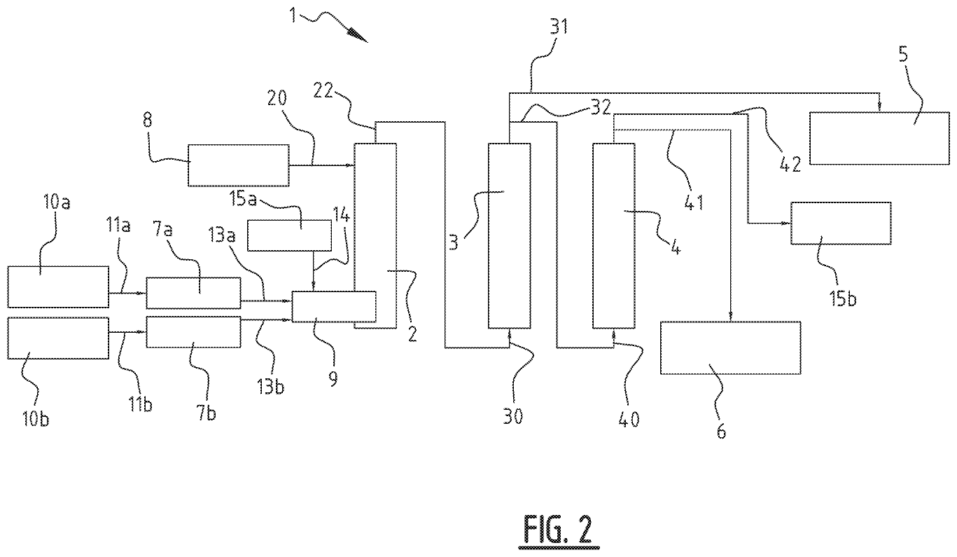

The embodiment shown in FIG. 2 differs from the one shown in FIG. 1 in that the water and the toluene each have a separate boiler (7a, 7b). Indeed, water and toluene are lead from their respective water and toluene tanks (10a, 10b) to a boiler (7a, 7b) through conduits (11a, 11b). In the boiler 7a, the water is brought to a temperature above its boiling point and pressurized to produce superheated water vapor. In the boiler 7b, the toluene is brought to a temperature above its boiling point and pressurized to produce superheated toluene vapor. The ensuing water and toluene vapor streams are led through their respective conduits (13a, 13b) to the sparger 9. In the sparger 9, the water and toluene vapor streams are thoroughly mixed and combined with the inert (N₂) gas stream, originating from the inert gas tank 15a through conduit 14.

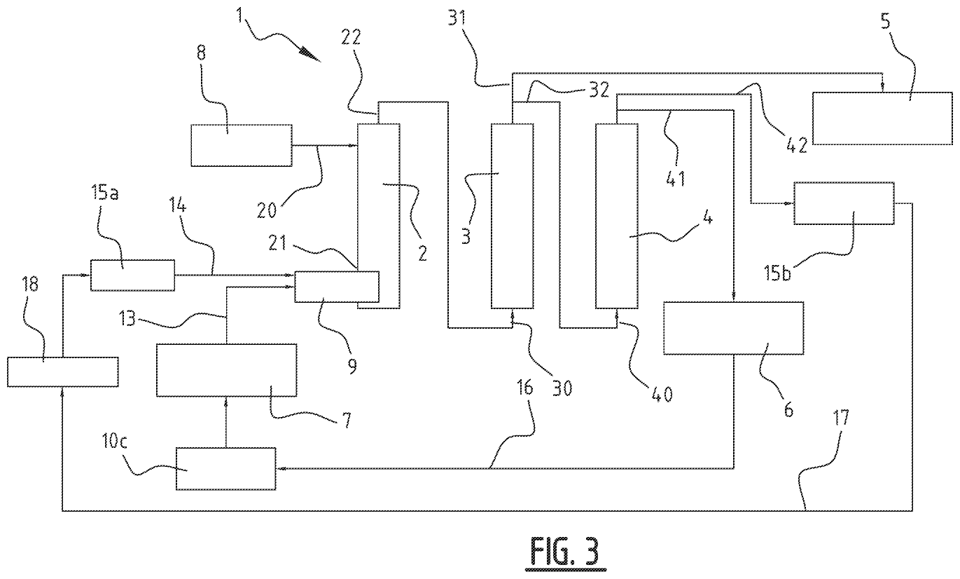

A third embodiment of the reactor system is shown in FIG. 3. The embodiment shown in FIG. 3 differs from the one shown in FIG. 1 in that the water/toluene mixture that is collected in solvent tank 6 after passing through the condensers (3, 4) is fed back to the mixing unit 10c through a feedback conduit 16. The water/toluene mixture is mixed again in the mixing unit 10c and led to the boiler 7 and to the sparger 9. It is then re-used as water/toluene vapor stream in the reactor 2. Also, the inert (N₂) gas collected in the inert gas tank 15b after passing through the condensers (3, 4) is fed back to the inert (N₂) gas tank 15a through a feedback conduit 17. Before entering the inert gas tank 15a, the inert gas is passed though a compressor 18 in which it is compressed to a suitable pressure. It is then re-used as inert gas stream in the sparger 9 and reactor 2.

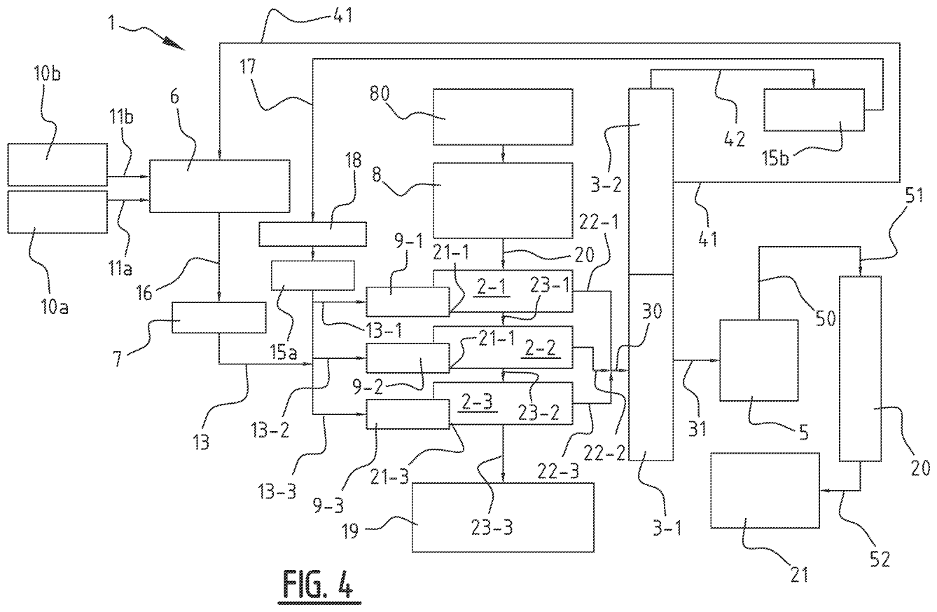

A fourth embodiment of the reactor system is shown in FIG. 4. This embodiment comprises a plurality of reactors (2-1, 2-2, 2-3) and the separating means comprise a multi-stage condensing unit (3-1, 3-2). The reactor system 1 in this embodiment comprises three stirred reactors (2-1-, 2-2, 2-3) that are provided in series. A melt 80 of PA6 is transported by first feeding means in the form of a gear pump or extruder 8 and enters a first stirred reactor 2-1 through an inlet 20, provided at a top portion of the first reactor 2-1. A bottom inlet 21-1 is provided to the first stirred reactor 2-1 through which an inert gas such as nitrogen gas (N₂) and superheated vapor are introduced in the first stirred reactor 2-1 via conduit 13-1. The introduction is preferably done through a first sparger 9-1, provided at a bottom portion of the first reactor 2-1. A bottom outlet 23-1 of the first reactor 2-1 connects to a top portion of a second reactor 2-2. A bottom inlet 21-2 is provided to the second stirred reactor 2-2 through which an inert gas such as nitrogen gas (N₂) and superheated vapor are introduced in the second stirred reactor 2-2. This is preferably done through a second sparger 9-2, provided at a bottom portion of the second reactor 2-2. A bottom outlet 23-2 of the second reactor 2-2 likewise connects to a top portion of a third reactor 2-3. A bottom inlet 21-3 is again provided to the third stirred reactor 2-3 through which an inert gas such as nitrogen gas (N₂) and superheated vapor are introduced in the third stirred reactor 2-3. This is preferably done through a third sparger 9-3, provided at a bottom portion of the third reactor 2-3.

The caprolactam contained in a vapor stream exits each stirred reactor (2-1, 2-2, 2-3) at a top portion thereof through exits (22-1, 22-2, 22-3). The caprolactam streams then enter a first stage of the multi-stage condenser (3-1, 3-2) through inlet 30 and leave the first stage 3-1 of the condenser through an outlet 31 that connects to the caprolactam product tank 5. Solvent, inert gas and other impurities enter the second stage 3-2 of the condenser. Condensed solvent exits the second stage 3-2 of the condenser through an outlet 41 and is collected in the solvent tank 6. Inert gas exits the second stage 3-2 of the condenser through an outlet 42 and is collected in an inert gas tank 15b.

Residual solids, such as other non-depolymerizable polymers, for instance polyolefins, that may be present in the PA6 polymer waste stream, and inert fillers such as glass fibers, talc and other mineral fillers exit the third reactor 2-3 though a bottom outlet 23-3 thereof and are collected in a suitable storage tank 19.

In the embodiment shown in FIG. 4, water and toluene are led from their respective water and toluene tanks (10a, 10b) to the solvent tank 6, where it is combined with solvent that has exited the second stage 3-2 of the condenser and is recovered from there through conduit 41. The fresh and reused water and toluene are then fed to the boiler 7 through conduit 16. In the boiler 7, the water/toluene mixture is brought to a temperature above the highest boiling point of the two constituents and pressurized to produce superheated vapor and the ensuing water/toluene vapor is led through conduit 13 and further through conduits (13-1, 13-2, 13-3) to each sparger (9-1, 9-2, 9-3). An inert gas such as N₂ that originates from inert gas tank 15a is also fed to each sparger (9-1, 9-2, 9-3) through conduits (13-1, 13-2, 13-3). The water/toluene vapor mixture and the inert gas combine in each sparger (9-1, 9-2, 9-3) and are introduced in each reactor (2-1, 2-2, 2-3) under turbulent conditions.

Also, the inert (N₂) gas collected in the inert gas tank 15b after passing through the multistage condenser (3-1, 3-2) is fed back to the inert (N₂) gas tank 15a through a feedback conduit 17. Before entering the inert gas tank 15a, the inert gas is passed though a compressor 18 in which it is compressed to a suitable pressure. It is then re-used as inert gas stream in the spargers (9-1, 9-2, 9-3) and the reactors (2-1, 2-2, 2-3).

The advantage of using a plurality of reactors (2-1, 2-2, 2-3) resides in the fact that the caprolactam vapor stream is contacted with a fresh inert gas/water/toluene vapor mixture in each reactor (2-2, 2-2, 2-3). Further, a counter-flow transport of the water/toluene vapor mixture with respect to the PA6 polymer waste stream and possibly the non-depolymerizable solids, is also beneficial to the depolymerization.

As shown in FIG. 4, the continuous reactor system 1 may also comprise a purification condensing unit 50 for obtaining a purified caprolactam. The purification condensing unit 50 connects to the crude product tank 5 though an outlet 50 and to a purified product tank 21 through a bottom outlet 52.

The reactor 2 or reactors (2-1, 2-2, 2-3) preferably are provided with suitable stirring or mixing means 23 to improve transport and distribution of the PA6 melt. The preferred sparger or spargers are instrumental in creating turbulent mixing conditions in the reactor(s), which is enhanced by the hydrocarbon added to the water. Suitable mixing means are known to one skilled in the art and may easily be implemented.

According to the method and reactor system of the present invention, caprolactam is formed by contacting the PA6 waste with a superheated vapor stream comprising a hydrocarbon/water mixture at elevated temperatures and pressures. With “superheated vapor” is meant in the context of the present disclosure vapor that is heated to a temperature substantially higher than the temperature at which condensation to liquid would take place at the pressure used to create said vapor stream.

The PA6 waste may be of any origin, such as carpet with face fiber of nylon 6, and possibly a backing of polypropylene and/or rubber latex. Such a feedstock is typically shredded and fed to the reactor 2 as a melt through the inlet 20. This feeding may be achieved by any means known in the art, such as by using an extruder and/or a gear pump 8, operated at a temperature above the melting temperature of PA6. Extruders, and gear pumps in particular, further allow relatively high pressures to be developed in the melt, such as up to 250 bar.

The contact of the polycaprolactam waste stream with the superheated vapor in the reactor 2 or reactors (2-1, 2-2-, 2-3) requires the reactor vessel(s) to withstand the relatively high temperature and pressure conditions. An additional advantage of the presently claimed method is that it does not require the use of corrosive catalysts, such as acids, in particular acetic and phosphoric acid, according to the prior art. A stainless steel reactor vessel is therefore adequate for the purpose.

A good contact between the superheated vapor stream and the polycaprolactam waste in the reactor 2 is essential for an effective operation. In a preferred method therefore, the superheated vapor is introduced in the reactor 2 through a sparger and sparged using a plurality of inlets, such as when using a vapor distributor. As claimed, improved contact between the PA6 melt and the vapor stream is achieved by including mechanical agitation in the reactor 2, for instance by using a combination of rotating paddles and static fins. The reactor 2 may also be divided into several stages by means of baffles, and means provided for mechanical agitation in each stage.

As to the desired reactor temperatures, suitable temperatures are between 250° C. and 300° C., with an optimal temperature range of 280° C.±10° C. Higher temperatures may actually increase the risk of decomposition and ammonia formation. The heat is provided to the reactor 2 or reactors (2-1,2-2, 2-3) by means of the superheated vapor entering the reactor 2. Additional heat may be provided through introducing heated polycaprolactam waste feed, and/or through heating means provided in the wall of the reactor(s) 2.

As to the desired reactor pressure, this pressure may be regulated by means of a back-pressure regulator for instance. This may increase the superheated vapor stream mixture to remain in the reactor 2 or reactors (2-1, 2-2, 2-3) longer. An optimum gauge pressure range appears to be in the range of 20 barg ±5 barg at 280° C. However, even lower pressures may be suitable.

As to the geometry of the reactor(s), a suitable reactor vessel is cylindrical with an aspect ratio of 1:10 (diameter: height). Other aspect ratios however may also be used. It should be noted that more than one reactor vessel provided in series may be used also.

In the continuous method claimed, both the PA6 waste and the superheated vapor are fed to the reactor 2 or series of reactors (2-1, 2-2, 2-3) continuously. In the embodiments shown, caprolactam is recovered overhead. It may be necessary to discharge some polymer melt from the bottom of the reactor 2 or third reactor 2-3, this polymer melt being a mixture of several degradation products and possibly also non-PA6 materials that were present in the waste feed stream.

To increase the potential caprolactam concentration in the overhead, the superheated hydrocarbon/water vapor stream is preferably countercurrent to the flow of the molten PA6. This can be achieved by feeding the PA6 at a side or top portion of the reactor 2 or first reactor 2-1 when employing a series of reactors (2-1, 2-2, 2-3) and feeding the vapor under pressure from a bottom portion of the reactor 2 or of each reactor (2-1, 2-2, 2-3).

The composition of the vapor stream mixture comprises a hydrocarbon/water mixture, most preferably a water/toluene mixture. The inventive idea behind this is that by using such a water/toluene mixture, the reaction balance in the reactor(s) is shifted more favorably towards promoting caprolactam formation and also increases the kinetics thereof. It should be noted that the hydrocarbon added to the water does not substantially act as a catalyst, contrary to the function of the added water. The hydrocarbon is a mere process additive.

The use of the hydrocarbon/water mixture was shown to make a higher total mass flow possible, probably due to the significantly lower specific heat capacity of the hydrocarbon, such as toluene. Therefore, replacing a substantial part of the water with hydrocarbon may lower the overall energy consumption of the process. In fact, it has turned out that the presence of a relatively small amount of water catalyst in the reactor(s) is sufficient for the depolymerization reaction to proceed swiftly.

The caprolactam may be separated from the other components of the vapor stream, for instance by sending the caprolactam-containing vapor stream from the reactor 2 or series of reactors (2-1, 2-2, 2-3) overhead to the first partial condenser 3 or multi-stage condenser (3-1, 3-2) to obtain a condensate containing caprolactam. The caprolactam obtained from this condensate may be further purified by known techniques, such as distillation, crystallization and the like. The purified caprolactam may then be used to make polycaprolactam using known methods.

It turns out that the use of a hydrocarbon/water mixture, and preferably a toluene/water mixture, actually also increases the caprolactam recovery efficiency in the first condenser 3 or multistage condenser (3-1, 3-2), even though relatively more hydrocarbon, preferably toluene, is also condensed.

The solvent/inert gas mixture is send to the second condenser 4 or second stage of the multistage condenser (3-1, 3-2) which separates purified solvent from the inert gas stream. The inert gas such as N₂ exiting the second condenser 4 or second stage of the multistage condenser (3-1, 3-2) may be recycled and fed again to the reactor 2 or series of reactors (3-1, 3-2, 3-3).

The following examples illustrate various preferred embodiments of the invention. Comparative Experiments are according to the state of the art, using water or a water/acetic acid mixture.

### Examples 1-5 and Comparative Experiments a-D

For all Examples 1-5, PA6 melt was charged via an extruder to a 100 L cylindrical stainless steel reactor. The reactor was agitated by means of rotating paddles that ensured turbulent mixing conditions, at least in a lower half of the reactor. The rotating rate of the rotating paddles was 500 RPM in all Examples. The reactor vent was connected via a pressure relieve valve to a first condenser. Superheated vapor comprising a mixture of toluene and water was blown through the reactor from the bottom at a rate of 1 kg/min via a sparger at the bottom of the reactor. Also, an inert gas (N₂) was fed to the reactor. Caprolactam containing vapor was drawn overhead and led to the first condenser. The gauge pressure and temperature in the reactor were maintained at about 20 barg and 280° C. respectively.

The temperature in the first condenser was maintained at about 130° C. A caprolactam/solvent mixture (95/5) came out of the first condenser, and caprolactam was led to the crude product tank of 200 L volume. A solvent flow of about 1 kg/min entered the second condenser which was cooled to about room temperature and N₂ gas was stripped off. The solvent was then led from the solvent tank to a boiler to produce the superheated vapor at a temperature of 280° C. and a gauge pressure of 30 barg.

The Comparative Experiments were carried out in the same manner as Examples 1-5, except for the composition of the solvent mixture. Comparative Experiments A and B use a mixture of water and acetic acid, whereas Comparative Experiment C uses water only. Comparative Experiment D, although using a toluene/water mixture did not use the rotating paddles in the reactor.

The results in terms of caprolactam (CL) yield are shown in Table 1.

TABLE 1
caprolactam (CL) yield for various solvents

| | | | | | Toluene/ | | |
|---|---|---|---|---|---|---|---|
| | | | | Acetic | water | | % yield |
| Example | PA6 | Toluene | Water | acid | ratio | Color | CL |
| Comp A | 22.4 | — | 40.2 | 364.8 | 0:100 | Opaque | 15.9 |
| Comp B | 22.4 | — | 364.9 | 40.5 | 0:100 | Opaque | 45.6 |
| Ex. 1 | 22.6 | 239.3 | 4.3 | 35.4 | 98:2 | Transp. | 53.9 |
| Ex. 2 | 22.6 | 233.4 | 36.9 | 4.1 | 86:14 | Transp. | 83.7 |
| Ex. 3 | 22.6 | 236.5 | 40.7 | — | 85:15 | Transp. | 85.8 |
| Ex. 4 | 22.4 | 234.3 | 40.5 | — | 85:15 | Transp. | 87.7 |
| Comp C | 22.4 | — | 350.2 | — | 0:100 | Opaque | 71.1 |
| Comp D | 22.4 | 234.4 | 40.4 | — | 85:15 | NA | 51.4* |
| Ex. 5 | 22.5 | 149.8 | 145.0 | — | 50:50 | Transp. | 75.4 |

*in Comparative Experiment D, the reactor was not stirred

At a reaction temperature of 280° C. there was a substantially complete conversion of PA6 and residual polymer could hardly be observed. Experiments carried out a lower reaction temperature of 250° C. for instance, showed a clear residual polymer to be present in the reactor vessel. Experiments carried out above 300° C. showed a substantial amount of decomposed ACA intermediate and concurrent ammonia formation.

The Examples show that a moderate reaction temperature of 280° appears to be enough to achieve a caprolactam yield of 86-88%. We note here that the yield can theoretically never be 100% due to reaction balance and residue of dimers and oligomers. The Examples also show that a higher water concentration generally negatively affects the caprolactam yield since it is less favorable to the reaction balance (and also more ACA intermediate remains). Since caprolactam is thermodynamically more stable than ACA, in a 100% water mixture the yield may reach 71% but not higher (Comparative Experiment C).

### Examples 6-10 and Comparative Experiments E-H

The above procedure for examples 1-5 and Comparative Experiments A-D was repeated at other reactor temperatures, reaction times, and for different hydrocarbons including benzene, o-xylene and iso-octane. The results are shown in Table 2.

TABLE 2
caprolactam (CL) yield for various solvents

| | | wt % | | Reaction | Temper- | % |
|---|---|---|---|---|---|---|
| | Hydro- | hydro- | wt % | time | ature | yield |
| Example | carbon | carbon | Water | (min) | (° C.) | CL |
| Ex. 6 | toluene | 85.2 | 14.8 | 90 | 280 | 89.3 |
| Ex. 7 | toluene | 85.1 | 14.9 | 60 | 280 | 85.9 |
| Ex. 8 | toluene | 70.4 | 29.6 | 60 | 280 | 72.8 |
| Ex. 9 | toluene | 94.8 | 5.2 | 60 | 280 | 74.5 |
| Ex. 10 | benzene | 85.1 | 14.9 | 60 | 280 | 82.5 |
| Comp E | iso-octane | 85.2 | 14.8 | 60 | 270 | 48.6 |
| Comp F | iso-octane | 85.2 | 14.8 | 60 | 280 | 48.8 |
| Comp G | iso-octane | 85.2 | 14.8 | 180 | 270 | 54.5 |
| Comp H | toluene | 85.2 | 14.8 | 60 | 250 | 9.6 |

From the above description, one skilled in the art can easily ascertain the essential characteristics of this invention and may make changes and modifications to the disclosed embodiments without departing from the spirit and scope thereof, as claimed in the appended claims.

1. A continuous method for depolymerizing polycaprolactam waste into caprolactam, the method comprising the steps of:
a) feeding a melt of the polycaprolactam waste and an inert gas to a reactor in a continuous manner;
b) feeding water and a hydrocarbon having a boiling point at atmospheric pressure of between 80° C. and 270° C. to an inlet of the reactor and contacting the polycaprolactam waste with superheated steam of the water/hydrocarbon mixture at a temperature of between 260° C. and 300° C. and at a gauge pressure from 1 barg to 70 barg;
c) creating turbulent mixing conditions in the reactor;
d) forming a caprolactam-containing vapor stream in the reactor which exits the reactor at an outlet;
e) separating caprolactam from the exited caprolactam-containing vapor stream by partial condensation; and
f) collecting the caprolactam from said condensed caprolactam-containing vapor stream; wherein
said water/hydrocarbon mixture comprises at least 1 wt. % and at most 40 wt. % water, relative to the total weight of the water/hydrocarbon mixture, and
the hydrocarbon is aromatic in that it comprises benzene and/or benzene derivatives having from one to six substituents attached to the central benzene core.

2. Method as claimed claim 1, wherein the hydrocarbon has a boiling point of less than 200° C., more preferably of less than 160° C., even more preferably of less than 140° C., and, most preferably around the boiling point of water at atmospheric pressure.

3. Method as claimed in claim 1, wherein the hydrocarbon has an auto-ignition temperature of more than 200° C., more preferably of more than 300° C., even more preferably of more than 400° C.

4. Method as claimed in claim 1, wherein the hydrocarbon comprises an alkylaromatic hydrocarbon.

5. Method as claimed in claim 4, wherein the alkyl of the alkylaromatic hydrocarbon comprises methyl, ethyl, propyl, butyl or pentyl, and the alkylaromatic hydrocarbon preferably comprises toluene and/or xylene and its isomers.

6. Method as claimed in claim 1, wherein the hydrocarbon is bio-based.

7. Method as claimed in claim 1, wherein said temperature is between 270° C. and 290° C.

8. Method as claimed in claim 1, wherein said gauge pressure is from 3 to 60 barg, more preferably from 5 to 40 barg, and most preferably from 10 to 30 barg.

9. Method as claimed in claim 1, wherein said water/hydrocarbon mixture comprises at most 30 wt. % water, even more preferably at most 20 wt. % water, and most preferably at most 15 wt. % water, relative to the total weight of the water/hydrocarbon mixture.

10. Method as claimed in claim 1, wherein said water/hydrocarbon mixture comprises at least 2 wt. % water, even more preferably at least 4 wt. % water, and most preferably at least 5 wt. % water, relative to the total weight of the water/hydrocarbon mixture.

11. Method as claimed in claim 1, wherein said contacting occurs in counter current flow with the superheated steam in a vertical tubular reactor, preferably with an open head.

12. Method as claimed in claim 1, wherein light fractions of the separating means that comprise the inert gas and the water/hydrocarbon mixture are fed back to the second feeding means for the inert gas and the third feeding means for the water and hydrocarbon respectively.

13. A continuous reactor system for depolymerizing polycaprolactam waste into caprolactam, the reactor system comprising:
first feeding means for feeding a melt of the polycaprolactam waste to a reactor in a continuous manner;
second feeding means for feeding an inert gas to the reactor in a continuous manner; third feeding means for feeding water and a hydrocarbon having a boiling point at atmospheric pressure of between 100° C. and 270° C. to an inlet of the reactor and creating turbulent mixing conditions in the reactor;
heating means for heating the reactor to a temperature of between 250° C. and 300° C.; and pressure means for pressurizing the reactor to a gauge pressure from 1 barg to 70 barg, such that the polycaprolactam waste is contacted with superheated steam of a mixture of the water and the hydrocarbon in the reactor;
mixing means provided in the reactor for transporting and distributing the polycaprolactam melt;
a reactor outlet for exiting a caprolactam-containing vapor stream formed in the reactor;
separating means for separating caprolactam from the exited caprolactam-containing vapor stream by partial condensation; and
collecting means for collecting the caprolactam from said condensed caprolactam-containing vapor stream;
wherein light fractions of the separating means that comprise the inert gas and the water/hydrocarbon mixture are fed back via feedback conduits to the second feeding means for the inert gas and the third feeding means for the water and hydrocarbon respectively, and wherein the third feeding means comprise a mixing unit for mixing the water and the hydrocarbon, and a boiler connected to said mixing unit.

14. Continuous reactor system as claimed in claim 13, wherein the first feeding means comprises an extruder and/or gear pump.

15. Continuous reactor system as claimed in claim 13, wherein the second and third feeding means are combined into a sparger for feeding the inert gas and a mixture of the water and hydrocarbon to the reactor, preferably at a bottom part thereof.

16. Continuous reactor system as claimed in claim 13, wherein the third feeding means comprise a boiler for each of the water and the hydrocarbon.

17. (canceled)

18. Continuous reactor system as claimed in claim 13, comprising a plurality of reactors.

19. Continuous reactor system as claimed in claim 13, wherein the separating means comprise a multi-stage condensing unit.

20. Continuous reactor system as claimed in claim 13, wherein the collecting means for collecting the caprolactam from said condensed caprolactam-containing vapor stream connect to a purification condensing unit for obtaining a purified caprolactam.
